(19) United States
(12) Patent Application Publication
(10) Pub. No.: US 2014/0102983 A1
(43) Pub. Date: Apr. 17, 2014

(54) DIALYSIS OPTIMIZING METHOD

(71) Applicant: B. BRAUN AVITUM AG, MELSUNGEN (DE)

(72) Inventors: JOERN MEIBAUM, BAUNATAL (DE); CHRISTOF STROHHOEFER, KASSEL (DE)

(21) Appl. No.: 14/053,246

(22) Filed: Oct. 14, 2013

(30) Foreign Application Priority Data
Oct. 16, 2012 (DE) 10 2012 109 858.1

(51) Int. Cl. A61M 1/16

(52) U.S. Cl. CPC A61M 1/16
USPC 210/647; 210/96.2

(57) ABSTRACT

A method as well as an apparatus is disclosed for determining the efficiency of a currently performed kidney replacement therapy on the dialysis side making use of a dialysis machine which in a first step is operated in a hemodialysis or hemodiafiltration process and in a second step is sequentially changed over to a hemofiltration process or is changed over to a sequential mode in which merely a flow from the blood compartment via the semipermeable membrane to the dialysis fluid compartment of the dialyser is generated in which, according to a third step, a sensor for determining or measuring the concentration at least of uremic toxins in the saturated dialysate or ultra-filtrate which is connected downstream of a dialyser on the dialysis side outputs corresponding measuring signals that are representative of the current concentration at least of uremic toxins in the blood to a calculation or determination unit.

### CROSS REFERENCE TO RELATED APPLICATIONS

This application claims priority to German application DE 10 2012 109 858.1 filed Oct. 16, 2012, the contents of such application being incorporated by reference herein.

### FIELD OF THE INVENTION

The present invention relates to a method as well as an apparatus for determining the efficiency of a currently carried out kidney replacement therapy and of the therapeutic apparatus.

### BACKGROUND OF THE INVENTION

Patients suffering from renal insufficiency have to have uremic toxins accumulating in the patient's body over the time removed by means of an extracorporeal kidney replacement therapy, hereinafter referred to as dialysis treatment, if or as long as no kidney transplantation is envisaged. By such dialysis treatment blood is collected from the patient, is guided through an extracorporeal purifying device—the dialyser—and is subsequently returned into the patient's body. In the dialyser the blood to be purified then gets in contact with a rinsing solution—the fresh dialysis fluid—via a semipermeable membrane, which results in an exchange of substance. The dialysate now enriched with uremic toxins is then rinsed and discarded.

For evaluating a dialysis therapy the efficiency of the afore-described dialysis treatment and the dialysis machine used is of salient importance. Therefore, in the past various methods have been developed and employed which permit judging the therapy and the condition of the machine after respective completion thereof. More recently, the focus has been put on determining the efficiency of a dialysis therapy already during the treatment itself so as to be able to manually intervene and possibly make corrections.

In order to ensure an efficient dialysis therapy therefore the K*t/V (urea) model, as it is called, was developed, wherein

K=purifying capacity of the dialyser (ml/min),

T=dialysis time (min) and

V=urea distribution volume in the patient (ml).

Moreover, also the urea reduction rate (URR, in %) was introduced as a suited means for determining the urea reduction in human bodies.

The NCDS (National Cooperative Dialysis Study) as well as the HEMO (hemodialysis) study prove after examination of a plurality of dialysis patients that the mortality and morbidity in ESRD (end-stage-renal-disease) patients strongly correlates with the afore-mentioned K*t/V value (i.e. the dialysis dosage). Data from said studies for example result in the fact that in the directives for carrying out a suitable dialysis therapy a treatment is deemed to be efficient only when a value K*t/V≧1.2 is reached.

In this context, it is referred to the fact that the judgment of the dialysis dosage by means of a K*t/V value is a relative assessment, similar to the afore-mentioned URR. However, this method does not permit any absolute statement about the actually removed amount of substance.

In K*t/V determination it is difficult to determine the purifying capacity K and the urea distribution volume V, due to the urea kinetics. For example, V can be determined more or less exactly by bio-impedance measuring, empirical estimations or by calculation by means of the known urea kinetics model (UKM). K can be established by determining urea in the blood of the patient before and after a dialysis therapy or by determining the change of conductivity at the dialyser input and at the dialyser output, resp., of the dialysate cycle.

Taking blood samples constitutes the reference method. After taking samples and settling with the UKM or the Daugirdas formula known per se, a K*t/V value based on a compartment as basis of calculation (spKTV) is obtained. Furthermore there exists a formula further developed by Daugirdas which now provides an equilibrated K*t/V (eKTV) value which also takes the urea generation as well as the rebound into consideration. This known method has one crucial drawback, though:

The evaluation of the dialysis dosage spK*t/V and eK*t/V again is not possible before the dialysis treatment is completed, because still the urea blood values of the patient are required.

Conductivity measuring methods are finally based on the assumption that the sodium purifying capacity of the dialyser is almost identical to that of urea. The ratio of

“sodium conductivity” to “sodium concentration”

is moreover temperature-dependent, however. Since the temperature change by tempering the dialysate is only small, however, it is assumed to be constant in the temperature range considered (e.g. 37° C.+/−2° C.). Therefore it is possible to determine the purifying capacity of urea by measuring the purifying capacity of sodium. The advantages of this method reside in the fact that they can be implemented relatively easily and are also cost-efficient. It offers K*t/V measurements during the dialysis therapy and thus permits correcting interventions by an operator already before the treatments are completed.

However, also conductivity measurements according to the foregoing description have drawbacks:

On the one hand, the method results in the fact that sodium is administered to the patient inadvertently/enforcedly, on the other hand in practice measuring intervals of a minimum of 20 min were reached, which effectively means approx. 12 measurements within an average dialysis therapy of 4 hours. Finally conductivity measuring methods are error-prone, as the measurement is influenced by other substances having ionic properties which change/influence the total conductivity and thus impede/falsify the calculation of the sodium concentration.

Another method of determining the dialysis efficiency is the direct measurement of the uremic substances (e.g. urea) in the used dialysate.

In this context, there are two possibilities both of which bypass the direct determination of the purifying capacity and of the urea distribution volume, respectively:
A first possibility consists in assuming the change of the urea concentration in the blood as well as in the used dialysate to be linear. Therefore, the gradient of the curve obtained after having determined the natural logarithm of the absorbed concentrations is identical in the dialysate and in the blood. The problem of this method becomes clear, however, when during therapy a reduction of the purifying capacity occurs e.g. by so called recirculation or by blocking of the dialyser, for this entails an increased gradient and thus a seemingly improved K*t/V value, although this is not the case.
A second possibility is described especially in the state of the art according to EP 0 986 410. In this document a good correlation of the “whole body clearance (wbK*t/V) and the Daugirdas K*t/V is found. This approach assumes the purifying capacity of the dialyser to be constant within predetermined time intervals and thus calculates the K*t/V value quasi “in reverse”. Although this second possibility is safer per se than the afore-described first possibility, it is still based on the assumption of constant conditions within predetermined time intervals and therefore is not adapted to calculate the wbK*t/V value at the beginning of therapy.

The methods of determining concentrations of uremic toxins relate to urea sensors and UV spectrophotometry. The technical restrictions when using urea sensors reside in their stability and reusability, however.

It was further illustrated that by UV spectroscopy an excellent correlation with the concentration of uremic substances (e.g. uric acid, urea etc.) in the used dialysate can be made. Since by this method a measure for the concentration of uremic toxins in the used dialysate is provided, it is possible to represent the relative change of uremic toxins in accordance with a K*t/V value. This method also exhibits a drawback, though:

Reductions of the purifying capacity of the dialyser, e.g. by blocking, are not detected by this method. Although the purifying capacity would be drastically reduced, the indication of the K*t/V value is increased in such case. Moreover physiological changes in the patient can be misinterpreted during the dialysis therapy.

If a sudden/abrupt change of the concentration of uremic toxins in the patient's blood occurs e.g. by vasoconstriction, water absorption or other metabolic peculiarities, these changes are possibly misinterpreted (too positively or too negatively) by the relative approach of the K*t/V value and therefore do not reproduce the actual status of the patient. At present the concentration of a substance in the patient's blood therefore can be determined almost exclusively only by chemical analysis of the blood itself.

### DESCRIPTION OF THE RELATED ART

EP 2 005 982 B1 finally describes a method in which by suitable fluid guiding in the dialysis machine the concentration of uremic toxins in the dialysate reaches the same level as in the patient's blood. This is achieved by a circulation of the dialysate limited in time through the dialyser, while the blood continues being exchanged between the patient and the dialyser according to therapy. In doing so, saturation of the dialysate with uremic toxins takes place. The concentration of said toxins and the absorption coefficient in the analyzed wavelength caused by toxins in the blood eliminated in the dialyser thus can be directly measured in the dialysate.

### SUMMARY OF THE INVENTION

As already described in the foregoing, methods determining the dialysis dosage in real time have weaknesses in the field of interpreting physiological changes in the patient and changes of the dialyser properties (purifying capacity) with respect to the calculation of the K*t/V value. Since this value always describes the relative change, necessarily misinterpretations occur in this case. The dialysis dosage in accordance with a K*t/V value is either overestimated or underestimated in this case depending on the situation.

Another problem occurs in methods that establish the K*t/V value by taking blood samples on the basis of the urea concentration. They, too, determine merely the relative change of the urea concentration before and after a dialysis therapy and establish the K*t/V value on the basis of Daugirdas. Depending on the method, a single-compartment or double-compartment is assumed and approached as model function. Physiological changes in the patient, for example by food intake, or changes of the physiological condition are taken into account to a limited extent only.

The drawback of the described method for saturating the dialysate with the blood-side concentration of uremic toxins is its complicated implementation in a dialysis machine. For this purpose, the use of additional tube connections and appropriate valves as well as control units is required to change over to circulating operation which only permits saturation of the dialysate. In addition, the sensor by which the concentration is determined must be arranged at the corresponding circulating cycle which is complicated to realize on the machine side, because at the same time the volume of the circulating cycle has to be kept as small as possible. Accordingly, also compromises regarding the total measuring time have to be made in this case.

In view of the afore-described problems, an object of the present invention is to develop a measuring methodology which enables the concentration of uremic toxins to be determined in the blood of patients requiring dialysis on the dialysis side. It is an aim in this context that this measuring method can do without direct contact with the patient and thus cannot be considered to be interventional in this respect. The method according to aspects of the invention furthermore is to make use of the methods known on the dialysis side (applied in the dialysis machine) so as to measure the blood-side concentration of uremic toxins. Preferably this is to be carried out during a dialysis therapy without having to interrupt the same.

This object is achieved by a measuring method for determining uremic toxin concentrations comprising the features (method steps) of claim 1 as well as by a dialysis machine comprising the features of claim 7. Advantageous developments are the subject matter of the subclaims.

In accordance with an aspect of the present invention, thus a method of determining the efficiency of a kidney replacement treatment currently carried out on the dialysis side using a dialysis machine which is operated in a first step in a hemodialysis or hemodiafiltration process and in a second step is sequentially changed over to a hemofiltration process or is changed over to a sequential mode in which merely a flow from the blood compartment (dehydration of the patient) is generated via the semipermeable membrane to the dialysis fluid compartment of the dialyser, after which, according to a third step, a sensor connected to the dialyser on the dialysis side for determining or measuring the concentration of at least uremic toxins in the saturated dialysate (ultra-filtrate) outputs appropriate measuring signals to a calculating or determining unit which are representative of the current concentration at least of uremic toxins in the patient's blood.

By hemofiltration generally a method is understood in which the concentrations of water-soluble substances in the patient's blood and an excess of patient's fluid are corrected by unidirectional convective transport by means of ultra-filtration via the semipermeable membrane separating blood from dialysis fluid and at the same time ultra-filtrate is replaced with an approximately iso-osmolar substitution fluid at an appropriate rate so that the difference of the rates between ultrafiltration and addition of substitution fluid results in the net dehydration.

Preferably the change-over step and the following measuring step are performed at least at the beginning and preferably also during and at the end of a dialysis treatment. Moreover, upon completion of the measuring step the dialysis machine is to be changed back to the hemodialysis or hemodiafiltration process without a circulating process extending the time of dialysis being necessary.

Another aspect of the present invention relates to a dialysis machine comprising a measuring device for measuring and/or determining the concentration at least of uremic toxins in a patient's blood. This dialysis machine according to aspects of the invention comprises the following technical features:

A dialyser against the input side of which fresh dialysis fluid can flow from at least one supply pump and

at least one discharge pump connected to the output side of the dialyser so as to convey the dialysate contaminated at least with uremic toxins out of the dialyser.

In accordance with the invention, a valve means possibly comprising plural closing valves is provided which allows for a fluid communication of the at least one supply pump to the dialyser in a hemodialysis or hemodiafiltration mode of the machine and disconnects such fluid communication in a hemofiltration mode of the machine and instead opens a bypass channel to the dialyser, and at least one sensor means connected to the dialyser and configured to send, in the hemofiltration mode of the machine or in a sequential mode in which merely a flow from the blood compartment (dehydration of the patient) via the semipermeable membrane to the dialysis fluid compartment of the dialyser is generated, measuring signals to a calculating and/or determining unit which signals are indicative of the concentration at least of uremic toxins in the fluid mixture of ultra-filtrate from the blood and the dialysis fluid in the dialyser compartment of the dialyser (hereinafter simply referred to as “ultra-filtrate”) and are representative of the toxin concentration in the patient's blood.

In this context it is pointed out that in practice it can be measured in the ultra-filtrate only when the dialysis fluid compartment of the dialyser is filled with ultra-filtrate. The latter is formed only after several minutes, though. However, the process of enrichment with toxins at the measuring device usually is by far faster. The reason for this is the diffusive substance transport of the uremic toxins which is substantially faster due to the difference in concentration between blood and dialysis fluid than the convective substance transport.

As is known, hemodialysis, hemodiafiltration and hemofiltration are treatment methods (treatment modes) in which the blood is treated by means of diffusion and convection. In hemodialysis the blood is substantially treated by means of diffusion, while in hemofiltration the blood is treated by means of convection. Hemodiafiltration combines both, i.e. diffusion by the dialysis fluid flow in the dialysis fluid compartment of the dialyser and the substitution flow into the extracorporeal circulation. In the hemofiltration mode the intake to the dialysis fluid compartment of the dialyser is disconnected. The blood treatment is then carried out by the substitution flow into the extracorporeal circulation.

In the afore-mentioned sequential mode merely a flow from the blood compartment via the semipermeable membrane to the dialysis fluid compartment of the dialyser is generated, whereas in the hemofiltration mode substitution fluid is pumped into the extracorporeal circulation so that via the semipermeable membrane the sum consists of dehydration of the patient and the substitution flow.

Thus the relation of the flows is as follows:

The flow through the sensor means depends on the treatment mode. In hemodialysis it is the sum of dialysis fluid flow and ultra-filtration flow. In hemofiltration it is the sum of substitution flow and net dehydration. In hemodiafiltration it is the sum of dialysis fluid flow plus substitution flow and net dehydration. Finally in a sequential treatment mode it is the ultra-filtration flow or net dehydration.

It may preferably be provided that the sensor means is connected to a connecting line between the dialyser and the discharge pump upstream to the bypass channel. Alternatively or additionally the sensor means may be arranged downstream of the discharge pump.

On the basis of the foregoing features the following advantages can be achieved compared the known state of the art:
Simple implementation in all existing dialysis machines, as the blood-side concentration is provided, according to aspects of the invention, on the dialysate side by components already present in all machines, viz. a possibly additional ultra-filtration pump.
The discharge of uremic toxins is at no time completely interrupted as it is the case in a circulation phase. Instead it is changed over between a hemodialysis phase or hemodiafiltration phase and a hemofiltration phase and a sequential phase (sequentially changed), i.e. only established blood purification methods are employed so that no delay of the dialysis therapy is resulting herefrom.
In the known circulation method the blood-side concentration is reached only after a particular setting time determined by the rate of saturation. In the method suggested according to aspects of the invention the blood-side concentration is provided immediately upon transition to the sequential phase (sequential mode) in the fluid in the dialysis fluid compartment of the dialyser (filtration phase in the filtrate). This permits more rapid determination of the concentration in the fluid in the dialysis fluid compartment at the output of the dialyser (in the filtrate on the dialysate side).
The arrangement of the sensor for concentration measuring is less critical in the suggested case. Said sensor can be positioned basically anywhere in the drain of the dialysate/ultra-filtrate (for reasons of accuracy a position ahead of the discharge pump is to be preferred, however). Hence the realization of this method requires less complexity during assembly of the machine.

In other words, the subject matter of the current development thus relates to the realization of a method for judging the absolute concentration of uremic toxins in the blood of dialysis patients on the dialysis side and not on the blood/patient side. This is preferably realized by measuring optical properties in the used dialysate/ultra-filtrate during a sequential ultra-filtration phase. Moreover, it is possible by means of an extension of this method to determine the purifying capacity (clearance) of the dialyser.

As described already, the evaluation of a therapy by means of the relative dialysis dosage K*t/V is related with several weaknesses. By measuring a degree for determining the absolute concentration in the blood at the beginning of the dialysis therapy according to the present invention, however, the actually removed amount of uremic toxins in the used dialysate/ultra-filtrate can be taken as a basis for evaluating a dialysis therapy. This is referred to as the absolute amount of removed substances over a dialysis therapy. This method exhibits plural advantages:

On the one hand, changes of the purifying capacity by blocking of the dialyser are not misinterpreted, because only the actually removed amount of uremic toxins passing the dialyser is measured. This is also applicable to physiological changes in the patient. For if there is an increase in or decrease of uremic toxins in the blood and thus necessarily also in the used dialysate/ultra-filtrate, in this case, too, only the actually removed amount of substance is established, irrespective of the amount of substance already removed (not relative).

By the same method, which will be described in detail hereinafter, it is possible, on the other hand, to quantify the purifying capacity of the dialyser at any time, but mainly at the beginning of a dialysis therapy. This is reasonable also with respect to the evaluation of a dialyser before the actual treatment begins, above all in the case of reuse of used dialysers. Further the concentration of uremic toxins in the patient can be observed over a long period of time (several weeks/months) and thus the degree of progress of the disease can be noticed. The measuring devices used can be any type of sensors or methods allowing for the determination of absolute concentrations. In this context both urea sensors and UV measuring devices are mentioned by way of example. The preferred method in this invention is the use of UV spectrometry.

The measuring method described in the following can generally be performed at any time of a dialysis therapy. When it is used for characterizing so called “clotting” or “blocking” of the dialyser, it is reasonable that it is carried out regularly during the dialysis therapy. In most cases it is sufficient, however, to apply the measuring method once at the beginning of the treatment.

At the beginning of a dialysis therapy the dialyser properties as well as the situation of the patient (shunt, nutritional condition) are most stable according to experience when considering the entire duration of a dialysis therapy. This situation can be used for absolutely determining the absorbance, for example, as a measure for the concentration of uremic toxins with the aid of a sequential phase according to aspects of the invention.

By activating the sequential phase the flow of dialysate via the dialyser is stopped (diverted) so that merely the ultra-filtrate (as defined before) reaches the measuring device (sensor) for determining absolute concentrations arranged in the dialysate drain. Due to the fact that substantially the convection as substance transport via the semipermeable membrane contributes to ultra-filtration, the concentration of uremic toxins is not changed, either. That is, the removed ultra-filtrate (actually fluid mixture as defined before) substantially contains the amount of uremic toxins which is also present in the blood. Since according to Lambert-Beer (1) the absorbance of aqueous fluids is suited as a measure for the concentration of optically active substances, the absolute, i.e. blood-side, concentration of uremic toxins can thus be determined on the dialysis side (via/by way of the ultra-filtrate). Upon completion of this measurement the sequential phase can immediately be deactivated again. In this way an interruption of the dialysis therapy is avoided, because also the sequential phase is suited for purifying the blood.

For the example of a UV measuring device the following is applicable:

$${A}_{UF} = log ( \frac{{I}_{0}}{{I}_{t}} ) or A = ɛ \cdot c \cdot l ⇒ A \sim c , if ɛ , l = const . \qquad ( 1 )$$

I₀ corresponds to the intensity of the UV measuring device with a fluid in which no uremic toxins are present. Iₜ, on the other hand, relates to the intensity measured at any time t. ε denotes the extinction coefficient that is equally known for known substances. Finally I represents the optical wavelength of the light of the UV radiation through the solution to be examined. The formula points out that initially the optical properties of non-used dialysate, i.e. without uremic toxins, have to be known. This measurement to determine I₀ can be carried out already during preparation of the dialyser. Finally A_{UF} represents the absorbance in the ultra-filtrate as a measure (representative) for the concentration of uremic toxins in the patient's blood.

Equation (1) first permits calculating the absorption coefficient α=ε*c by means of division through the optical path in an absorption measuring cell. The absorption coefficient describes the specific absorption at the measuring wavelength which the uremic toxins have in the blood. If moreover ε is known, e.g. because at the wavelength used only a particular substance is absorbed or because the specific share of a substance in the absorption coefficient can be isolated from a mixed signal by reason of common measuring techniques, the concentration of the substance results as additional information from the absorption coefficient.

The actually removed amount of substance over the dialysis therapy can now be quantified by the measure for the starting concentration of uremic toxins in the blood by continuously measuring used dialysate/ultra-filtrate. At the end of therapy, apart from the K*t/V calculation which is not influenced by this measurement, additional information is available which is not falsified by changes of the dialyser properties and of the patient's status. In contrast to this, those changes can even be recorded and when displayed on the screen can possibly inform about nuisances/changes and indicate required interaction of the nursing staff.

In order to allow for an as smooth and rapid measured data acquisition as possible the measuring device should be provided most closely to the dialysate drain of the dialyser (output side). However, any position downstream of the dialyser output to the drain is basically possible. In this context, a position in the drain ahead of a balancing means of the dialysis machine is to be preferred.

It is also possible that the performance of the sequential phase is stopped already before the ultra-filtrate reaches the sensor. For performing the described method a pulse sufficient for the measuring purposes is sufficient. For if the position of the sensor is known, the period of time until the pulse will reach the sensor can be estimated by the knowledge of the internal dialysate volume in the tubing systems as well as of the dialysate flow after stopping the sequential ultra-filtration phase. For this purpose, a pulse ranging approximately from 1 to 250 ml is required. Depending on the ultra-filtration rate during the sequential phase, for this a guessed period of few seconds to 5 minutes is required.

As an alternative, the ultra-filtration flow can also be used to convey the rinsing solution saturated already in the dialyser to the measuring device within a sequential phase. Assuming the fact that diffusive transports of substance take place by far more quickly than a convective transport of substance, the ultra-filtration flow would convey, apart from the convective share, also the share of the dialysate solution enriched by means of diffusion to the measuring device. Both effects are mutually dependent on each other, but they entail the same result:

The measuring solution conveyed to the measuring device by the ultra-filtration flow has the same concentration of uremic toxins as the blood.

The advantage of this type of realization is based on the fact that the ultra-filtration flow can be completely stopped in a period of time from the start of the sequential phase to the discharge of uremic toxins, which usually is less than ten minutes. After the end of the period a dialysis fluid flow (ultra-filtration flow) is set for conveying the dialysate saturated in the dialyser to the measuring device.

What is decisive to the measuring technique irrespective of the type of realization is the fact that the measured data acquisition is carried out as quickly as possible and as closely to the dialyser as possible. The local vicinity can be varied by the positioning of the measuring device in the dialysate circulation. The rate of the measured data acquisition is substantially reached by the level of the ultra-filtration flow as well as the dialysis-side volume.

This method according to aspects of the invention can also be applied to determine the purifying capacity of dialysers either for checking the values stated in the datasheet or for checking the purifying capacity of the dialyser for re-use. The way of proceeding is identical to the facts outlined in the preceding paragraphs:

The absorption coefficient of the saturated dialysate (ultra-filtrate) constitutes a measure for the actual concentration of uremic toxins in the blood of patients requiring dialysis. Upon completion of measurement, the provided method of treatment can only be continued by repeatedly connecting the dialysis flow via the dialyser. If a specific blood flow is adjusted, the purifying capacity of the dialyser is appropriately set. Due to the purifying capacity only a certain part of uremic toxins migrates into the dialysate and is detected by the UV measuring device there. After evaluating the related absorbance the actual purifying capacity of the dialyser can be determined. To this effect, the blood flow (Q_{BF}) and dialysate flow (Q_{DF}) have to be taken into account.

With A_{UF} representing the absorbance (proportional to the concentration) of uremic toxins in the pure ultra-filtrate and A_{DF} represents the absorbance of uremic toxins in the used dialysate, the purifying capacity of the dialyser after change-over to the HD/HDF therapy is resulting as follows (2):

$$K [ \% ] = \frac{{A}_{DF}}{{A}_{UF}} \cdot \frac{{Q}_{DF}}{{Q}_{BF}} and for the current {Q}_{BF} \text{:} K [ ml \text{/} min ] = \frac{{A}_{DF}}{{A}_{UF}} \cdot {Q}_{DF} . \qquad ( 2 )$$

The purifying capacity can be measured at any time during the treatment. It is a prerequisite, however, that the absolute concentration of uremic toxins has been determined in the blood of the dialysis patient before.

As a consequence, there is disclosed a method and a device for determining the efficiency of a currently performed kidney replacement therapy on the dialysis side using a dialysis machine that is operated in a first step in a hemodialysis or hemodiafiltration process and in a second step is changed over to a sequential mode in which merely a flow from the blood compartment (dehydration of the patient) via the semipermeable membrane to the dialysis fluid compartment of the dialyser is generated, in which, according to a third step, a sensor connected to a dialyser on the dialysis side for determining or measuring the concentration at least of uremic toxins in the saturated dialysate (ultra-filtrate) outputs appropriate measuring signals representative of the current concentration at least of uremic toxins in the blood to a calculation or determination unit.

### BRIEF DESCRIPTION OF THE DRAWINGS

Hereinafter the invention will be described in detail by way of a preferred embodiment with reference to the accompanying figures in which

FIG. 1 shows the basic structure of a dialysis machine including a measuring device according to aspects of the present invention and

FIG. 2 exemplifies the course of the measuring signal in the case of sequential phases initiated according to aspects of the invention.

### DETAILED DESCRIPTION OF THE PREFERRED EMBODIMENTS

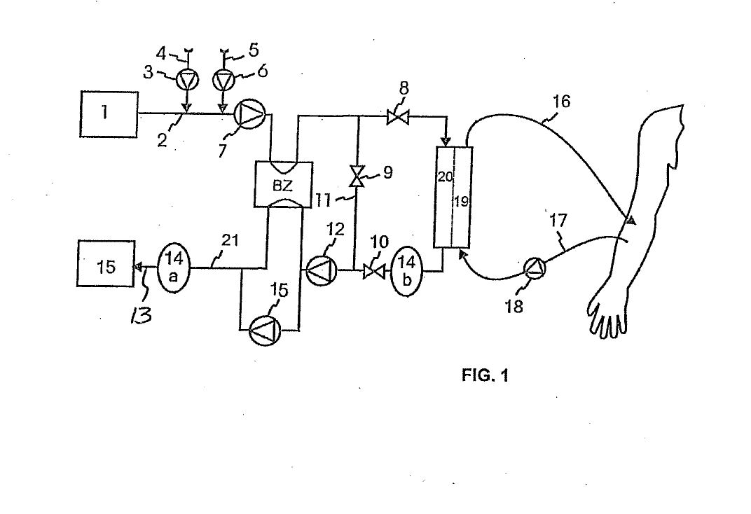

According to FIG. 1, the dialysis machine comprises at least one supply pump or flow pump dialyser input (FPE) 7 that supplies dialysis fluid via a supply line to a dialyser 19, at the input side thereof. The dialysis fluid is prepared in the present case by supplying water from a water treatment 1 via a water intake 2 of the supply pump 7, wherein a bicarbonate concentrate 4 is supplied by a bicarbonate pump 3 and an acid concentrate 5 is supplied by an acid pump 6 to the water. Moreover a balancing system BZ is interconnected in the supply line.

To an output side of the dialyser 19, 20 a drain line is connected leading to a first discharge pump or pump means at the dialyser output 12 which supplies used dialysis fluid (dialysate) to a collecting tank or dialysate drain 15 equally via the balancing system BZ. In parallel to the balancing system BZ a second booster pump 15 equally capable of conveying into the collecting tank/dialysate drain 15 is connected to the first discharge pump 12.

A bypass channel 11 is provided which is connected to the supply line and the drain line and, resp., to the inlet and outlet of the dialyser 19, 20 while short-circuiting the dialyser 19, 20 and in which an opening/closing valve 9 is interconnected. Furthermore another opening/closing valve 8 is disposed immediately ahead of the dialyser inlet downstream of the connecting point of the bypass channel 11.

A measuring device 14b adapted to the measuring of the concentration of uremic toxins is arranged directly at the dialyser outlet. A last opening/closing valve 10 disposed upstream of the connecting point of the bypass channel 11 is directly connected to said measuring device 14b. As an alternative or in addition to the measuring device 14b a like or similar measuring device 14a can also be disposed at the drain side of the booster pump 15.

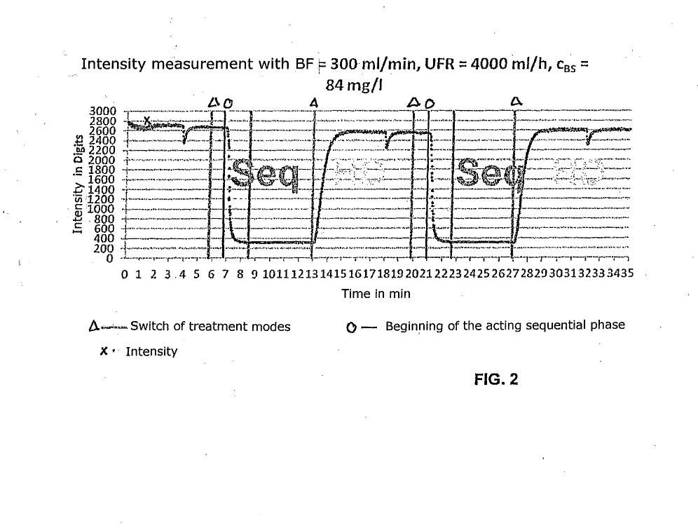

The function of the dialysis machine having the afore-described conceptual structure can be described as follows by way of FIG. 2:

During normal hemodialysis (which corresponds to the approximate treatment periods of 0-6 min, 13-20 min etc. according to FIG. 2) the valves 8 and 10 are opened and the valve 9 is closed. The freshly prepared water which is transformed into a physiologically well tolerable dialysis solution by the bicarbonate concentrate 4 and the acid concentrate 5 passes, while being driven by the pumps 7 and 12, first the balancing system BZ which ensures that the fluids flowing into and out of the dialyser 19, 20 are volumetrically identical. After having passed the balancing system BZ, the dialysis solution is pumped via the valve 8, the dialyser 19, 20 and the valve 10 by means of the pump 12 into the balancing system BZ again.

In the dialyser 19, 20 it enters into function contact with blood via a semipermeable membrane (not shown in detail), the blood passing the dialyser 19, 20 on the blood side thereof while being driven by a blood pump 18 in a blood supply line 17. The exchange of substance is usually performed by means of diffusion in this case.

In order to carry out measurement of the concentration of uremic toxins in the blood a sequential phase is started (which corresponds to the approximate treatment periods of 6-13 min and 20-27 min according to FIG. 2). For this, the valve 8 is closed and the valve 9 is opened, while the pumps 7 and 12 continue to convey without change. In this case the dialysis fluid does no longer flow via the dialyser 19, 20 but via the bypass channel 11. The booster pump 15, on the other hand, now additionally conveys (for generating a vacuum on the dialysis side in the dialyser) exclusively fluid (ultra-filtrate) from the dialyser 19, 20 which is withdrawn from the blood. The flow rate must be adapted according to the flow rate of the other pumps 7 and 12 to obtain the desired pressure difference in the dialyser.

When the saturated dialysis fluid (ultra-filtrate according to the above definition) now passes the measuring device 14b and/or 14a, the actual amount of uremic toxins in the patient's blood (which corresponds to the concentration of uremic toxins in the pure ultra-filtrate) can be measured on the dialysis side (not on the blood side). In this context, is referred to the fact that in the case of the measuring device 14a the ultra-filtrate is provided already mixed again with the still fresh dialysis fluid from the bypass channel 11, as this measuring device 14a is provided downstream of the bypass channel 11.

Upon completion of measurement the valve 8 opens again while the valve 9 closes so that the original dialysate flow can pass the dialyser again.

From FIG. 2 it can be inferred that, after changing over from e.g. a common dialysis therapy (hemodialysis (HD) or hemodiafiltration process (HDF)) to a sequential phase (or hemofiltration process (HF)) according to the above definition, the intensity of the UF measuring device decreases to a constant value which reflects the concentration of uremic toxins in the blood. The intensity value reached represents the concentration according to equation (1). As a matter of course, the method is formally applicable also to HF therapies; however the application is trivial in those cases as no change-over has to take place, because the therapy is carried out exclusively in sequential mode. Moreover, this type of therapy is used very rarely.

The absorbance A_{UF} measured by the method according to aspects of the invention can be exclusively used to determine the actual purifying capacity of the dialyser during a hemodialysis therapy by means of equation (2).

Moreover, it is possible and also recommendable for economic reasons to terminate the sequential phase already before the final measured value is reached. It is the idea to substantially accelerate the measuring process by carrying out e.g. merely a short-term, preferably one-minute sequential phase, before it is changed back to the HD therapy which per se is not long enough for the fluid to reach the sensor. Since the saturated fluid (ultra-filtrate sample) is already provided in the tubing system of the dialysis machine, it need not be waited until it has also reached the sensor. This will necessarily occur also after terminating the sequential phase by connecting the dialysate flow through the dialyser. In this case it is only necessary to ensure that the duration of the sequential phase is sufficient to convey a sufficiently large volume (fluid distance in the tubing) of blood-side fluid into the dialysate-side tubing system. In this context it has turned out that—dependent on the pump speed in the range of from 1-100 ml/min—after few seconds to minutes already the conveyed fluid volume is sufficient.

1. An apparatus for determining the efficiency of a currently performed kidney replacement therapy on the dialysis side of a dialysis machine which is adapted to be operated in a first operating step in a hemodialysis or hemodiafiltration process and in a second operating step is sequentially changed over to a hemofiltration process or is changed over to a sequential mode in which merely a flow from the blood compartment via the semipermeable membrane to the dialysis fluid compartment of the dialyser is generated in which, according to a third operating step, a sensor for determining or measuring the concentration at least of uremic toxins in the ultra-filtrate or in the saturated dialysis fluid which is connected downstream of a dialyser on the dialysis side outputs corresponding measuring signals that are representative of the current concentration at least of uremic toxins in the blood to a calculation or determination unit.

2. The apparatus according to claim 1, wherein the change-over operating step as well as the following measuring operating step are carried out at least at the beginning and preferably also during and at the end of a dialysis treatment.

3. The apparatus according to claim 1, wherein UV spectrometry is employed for measuring the concentration at least of uremic toxins.

4. The apparatus according to claim 3, wherein the absorbance is determined as a measure for the concentration at least of uremic toxins.

5. The apparatus according to claim 1, wherein after completion of the measuring operating step the dialysis machine is changed back to the hemodialysis or hemodiafiltration process.

6. The apparatus according to claim 1, wherein the dialysis machine is changed back to the hemodialysis or hemodiafiltration process already before completion of the measuring operating step, wherein the duration of the sequential mode or hemofiltration process is selected to be only such that a volume of blood-side fluid sufficient for measuring is conveyed into a dialysis-side tubing system leading to the sensor.

7. A dialysis machine comprising a measuring device for measuring and/or determining the concentration at least of uremic toxins in a patient's blood, comprising:
a dialyser against the input side of which fresh dialysis fluid can flow from at least one supply pump;
at least one discharge pump connected to the output side of the dialyser so as to convey the dialysate contaminated at least with uremic toxins from the dialyser;
a valve means which permits fluid communication of the at least one supply pump to the dialyser in a hemodialysis or hemodiafiltration mode of the dialysis machine and in a sequential mode in which merely a flow from the blood compartment via the semipermeable membrane to the dialysis fluid compartment of the dialyser is generated or in a hemofiltration mode of the machine disconnects said fluid communication and instead opens a bypass channel to the dialyser; and
at least one sensor means connected downstream of the dialyser and being configured to send, in the sequential mode or in the hemofiltration mode of the dialysis machine, measuring signals indicative of the concentration at least of uremic toxins in the saturated dialysate or ultra-filtrate to a calculation and/or determination unit.

8. The dialysis machine according to claim 7, wherein the bypass channel opens into a connecting line between the dialyser and the discharge pump.

9. The dialysis machine according to claim 8, wherein the sensor means is connected to the connecting line between the dialyser and the discharge pump.

10. The dialysis machine according to claim 9, wherein the sensor means is connected downstream of the discharge pump.

11. The dialysis machine according to claim 7, wherein UV spectometry is employed for measuring the concentration at least of uremic toxins.

12. A method for determining the efficiency of a currently performed kidney replacement therapy on a dialysis side making use of a dialysis machine comprising:
operating the dialysis machine in a hemodialysis, or hemodiafiltration process;
sequentially changing over to a hemofiltration process or changing over to a sequential mode in which merely a flow from a blood compartment via a semipermeable membrane to a dialysis fluid compartment of a dialyser is generated;
outputting with a sensor for determining or measuring the concentration at least of uremic toxins in the saturated dialysate or ultra-filtrate which is connected downstream of the dialyser on the dialysis side corresponding measuring signals that are representative of the current concentration at least of uremic toxins in the blood to a calculation or determination unit.

13. The method according to claim 12, wherein the change-over step as well as the subsequent outputting step are carried out at least at the beginning and preferably also during and at the end of a dialysis treatment.

14. The method according to claim 12, wherein UV spectrometry is employed for measuring the concentration at least of uremic toxins.

15. The method according to claim 14, wherein the absorbance is determined as a measure of the concentration at least of uremic toxins.

16. The method according to claim 12, wherein after completion of the outputting step, the dialysis machine is changed back to the hemodialysis or hemodiafiltration process.

17. The method according to claim 12, wherein the dialysis machine is already changed back to the hemodialysis or hemodiafiltration process temporally before completion of the outputting step, wherein the duration of the sequential process is selected to be only such that a volume of blood-side fluid sufficient for measuring is conveyed into a dialysis-side tubing system leading to the sensor.

18. The dialysis machine according to claim 11 wherein absorbance is determined as a measure for the concentration at least of uremic toxins.
